(19) United States
(12) Patent Application Publication
(10) Pub. No.: US 2001/0053801 A1
(43) Pub. Date: Dec. 20, 2001

(54) Inverse latexes based on white mineral oil, squalene or hydrogenated polyisobutene, cosmetic, dermocosmetic, dermopharmaceutical or pharmaceutical compositions containing them

(75) Inventors: Guy Tabacchi, Castres (FR); Jean Pierre Boiteux, Saix (FR); Chantal Amalric, Blan (FR); Nelly Michel, Maisons Alfort (FR); Paul Mallo, Chatou (FR)

Correspondence Address: YOUNG & THOMPSON, 745 SOUTH 23RD STREET 2ND FLOOR, ARLINGTON, VA, 22202

(73) Assignee: SOCIETE D'EXPLOITATION DE PRODUITS POUR LES INDUSTRIES CHIMIQUES SEPPIC

(21) Appl. No.: 09/849,339

(22) Filed: May 7, 2001

(30) Foreign Application Priority Data
May 5, 2000 (FR) 0 00 5791

(51) Int. Cl. C08K 3/00; A61K 31/74; C08L 31/00

(52) U.S. Cl. 523/105; 424/78.03; 424/78.31; 524/556

(57) ABSTRACT

Composition comprising an oil phase, an aqueous phase, at least one emulsifying agent of the water in oil (W/O) type, at least one emulsifying agent of the oil in water (O/W) type, in the form of an auto-inversible inverse latex comprising from 20% to 70% by weight, and preferably from 25% to 50% by weight, of a branched or reticulated polyelectrolyte, characterized in that said polyelectrolyte is either a homopolymer based on a monomer having a weakly acidic function, partially or totally salified, or a copolymer based on at least one monomer having a strongly acidic function, copolymerized either with at least one monomer having a strongly acidic function, or with at least one neutral monomer, and characterized in that the solvent constituting the oil phase is selected from white mineral oils, squalene or hydrogenated polyisobutene. Cosmetic, dermocosmetic, dermopharmaceutical or pharmaceutical composition including it.

The present application relates to inverse oil and water latexes, their preparation process and their application as thickeners and/or emulsifiers for products for skin care and hair care or for the production of cosmetic, dermocosmetic, dermopharmaceutical or pharmaceutical preparations.

Synthetic thickening polymers, in the form of inverse latexes, are described as being able to be used in the production of topical compositions, in French patent applications published under numbers 272511, 2733805, 2774688, 2774996 and 2782086, as well as in European patent application published under number EP 0 503 853.

However, certain of them sometimes give rise to reactions intolerable for certain sensitive skin.

This is why the applicant is interested in seeking new polymer emulsions, which will be better tolerated by the skin than those of the prior art.

The invention has for its object a composition comprising an oil phase, an aqueous phase, at least one emulsifying agent of the water in oil type (W/O), at least one emulsifying agent of the oil in water type (O/W), in the form of an auto-invertible inverse latex comprising 20 to 70% by weight, and preferably 25 to 50% by weight, of a branched or reticulated polyelectrolyte, said polyelectrolyte is either a homopolymer based on a monomer having a weakly acidic function partially or totally salified, or a copolymer based on at least one monomer having a strongly acidic function, copolymerized either with at least one monomer having a weakly acidic function, or with at least one neutral monomer, and characterized in that the solvent constituting the oil phase is selected from white mineral oil, squalene or hydrogenated polyisobutene.

The hydrogenated polyisobutene is sold in France by the company Ets B. Rossow et Cie under the name PARLEAM-POLYSYNLANE™. It is cited in: Michel and Irene Ash; Thesaurus of Chemical Products, Chemical Publishing Co., Inc. 1986, Volume 1, page 211 (ISBN 0 7131 3603 0).

Squalene is sold in France by the SOPHIM company under the name PHYTOSQUALAN™. It is identified in Chemical Abstracts by the number RN=111-01-3. It is a mixture of hydrocarbons containing more than 80% by weight of 2, 6, 10, 15, 19, 23-hexamethyl tetracosane.

By white mineral oil is meant, in the scope of the present invention, a white mineral oil according to the regulations FDA 21 CFR 172.878 and CFR 178.3620(a). The invention more particularly has for its object the composition as defined above, in which the white mineral oil constituting the oil phase is MARCOL™52. MARCOL™52 is a commercial oil corresponding to the definition of vaseline oils of the French CODEX.

According to a second particular aspect of the present invention, the solvent constituting the oil phase of the inverse latex, is hydrogenated polyisobutene.

According to a third particular aspect of the present invention, the solvent constituting the oil phase of the inverse latex, is squalene.

By a branched polymer is meant a non-linear polymer which has depending chains in a manner to obtain, when this polymer is placed in solution in oil, a strong interlocking meeting to very high low gradient viscosities.

By a reticulated polymer is meant a non-linear polymer present in the condition of a three-dimensional network insoluble in water, but swellable in water and thus leading to the obtention of a chemical gel.

The composition according to the invention can comprise reticulated and/or branched patterns.

By “emulsifying agent of the water in oil type”, is meant emulsifying agents having an HLB value sufficiently low to form water in oil emulsions such as tensioactive polymers sold under the name HYPERMER™ or such as sorbitan esters, like the sorbitan mono-oleate sold by the SEPPIC company under the mark MONTANE™ 80 or sorbitan isostearate sold by SEPPIC under the name MONTANE™ 70. There can also be included in these emulsifying agents, sorbitan oleate ethoxylated with 5 moles of ethylene oxide, sold by the SEPPIC company under the name MONTANOX™ 81.

By “emulsifying agent of the oil in water type”, are meant emulsifying agents having an HLB value sufficiently high to form oil in water emulsions such as ethoxylated sorbitan esters like sorbitan oleate ethoxylated with 20 moles of ethylene oxide, sold by the SEPPIC company under the name MONTANOX™ 80, caster oil ethoxylated with 40 moles of ethylene oxide sold by the SEPPIC company under the name SIMULSOL™ OL50, sorbitan laurate ethoxylated with 20 moles of ethylene oxide sold by the SEPPIC company under the name MONTANEX™ 20 or lauric alcohol ethoxylated with 7 moles of ethylene oxide, sold by the SEPPIC company under the name simulsol™ P7.

As emulsifying gents having a sufficiently high HLB value so as to from oil in water emulsions, there are also compounds of the formula (I):

R₁—O—[CH(R₂)—CH₂—O]ₙ-(G)ₓ-H (I)

in which R₁ represents a linear or branched hydrocarbon radical, saturated or unsaturated, comprising 1 to 30 carbon atoms, R₂ represents a hydrogen atom or alkyl radical having 1 or 2 carbon atoms, G represents the rest of a saccharide, x represents a decimal number comprising between 1 and 5, and n is equal either to zero or to a whole number comprised between 1 and 30.

By the rest of a saccharide, is meant for G, a bivalent radical resulting from the removal from a sugar molecule, on the one hand of a hydrogen atom from a hydroxyl group and on the other hand part of the anomeric hydroxyl group. The term saccharide designates particularly glucose or dextrose, fructose, mannose, galactose, altrose, idose, arabinose, xylose, ribose, gulose, lyxose, maltose, maltotriose, lactose, cellobiose, dextran, talose, allose, raffinose, levoglucan, cellulose or starch. The oligomeric structure (G)ₓ can be present in any isomeric form, whether an optical isomer, a geometric isomer or a position isomer; it can also represent a mixture of isomers.

In the formula (I) as defined above, the radical R₁—O—[CH(R₂)—CH₂—O]ₙ— is connected to G by the anomeric carbon so as to form an acetal function. The divalent group —[CH(R₂)—CH₂—O]ₙ— represents either a chain composed solely of ethoxyl groups (R₂═H), or a chain comprised only of propoxyl groups (R₂═CH₃), or a chain comprised both of ethoxyl groups and propoxyl groups. In this latter case, the fragments —CH₂—CH₂—O— and —CH(CH₃)—CH₂—O— are distributed in said chain, in a sequenced or random manner.

The number x, which represents in formula (I) the mean degree of polymerization of the saccharide, is more particularly comprised between 1 and 3, particularly between 1.05 and 2.5, and more particularly between 1.1 and 2.0 and preferably less than or equal to 1.5.

As emulsifying tensioactive agents having a sufficiently high HLB value to form oil in water emulsions, there are more particularly compounds of formula (I) as defined above, in which G represents the rest of the glucose or the rest of the xylose and/or in which n is equal to 0, and/or in which R₁ represents a radical comprising 8 to 18 carbon atoms and more particularly in which R₁ represents a radical selected from octyl, decyl, undecyl, dodecyl, tetradecyl or hexadecyl radicals, said radicals being linear or branched.

As examples of commercial products containing said compounds, there is for example:

SIMULSOL™ SL8, sold by the SEPPIC company, which is an aqueous solution containing between about 35% and 45% by weight of a mixture of alkyl polyglycosids consisting of between 45% by weight and 55% by weight of a compound of formula (I), in which G represents the rest of the glucose, x is equal to about 1.45, n is equal to 0 and R₁ represents a decyl radical, and between 45% by weight and 55% by weight of a compound of formula (I), in which g represents the rest of the glucose, x is equal to about 1.45, n is equal to 0 and R₁ represents an octyl radical;

SIMULSOL™ SL10, sold by the SEPPIC company, which is an aqueous solution containing between about 40% by weight and 50% by weight of a mixture of alkyl polyglycosids, consisting of about 85% by weight of a compound of formula (I), in which G represents the rest of the glucose, x is equal to about 1.45, n is equal to 0 and R₁ represents a decyl radical, about 7.5% by weight of a compound of formula (I), in which G represents the rest of the glucose, x is equal to about 1.45, n is equal to 0 and R₁ represents a dodecyl radical and about 7.5% by weight of a compound of formula (I), in which G represents the rest of the glucose, x is equal to about 1.45, n is equal to 0 and R₁ represents a tetradecyl radical;

SIMULSOL™ SL11, sold by the SEPPIC company, which is an aqueous solution containing between about 40% by weight and 50% by weight of a mixture of alkyl polyglycosids of formula (I), in which G represents the rest of the glucose, x is equal to about 1.45, n is equal to 0 and R₁ represents an undecyl radical; or

SIMULSOL™ SL26, sold by the SEPPIC company, which is an aqueous solution containing between about 40% by weight and 55% by weight of a mixture of alkyl polyglycosids consisting of about 70% by weight of a compound of formula (I), in which G represents the rest of the glucose, x is equal to about 1.45, n is equal to 0 and R₁ represents a dodecyl radical, about 25% by weight of a compound of formula (I), in which G represents the rest of the glucose, x is equal to about 1.45, n is equal to 0 and R₁ represents a tetradecyl radical at about 5% by weight of a compound of formula (I), in which G represents the rest of the glucose, x is equal to about 1.45, n is equal to 0 and R₁ represents a hexadecyl radical.

The strongly acidic function of the monomer in question is particularly the sulfonic acid function or the phosphonic acid function, partially or totally salified. Said monomer can be for example styrenesulfonic acid, partially or totally salified. It is preferably 2-methyl 2-[(1-oxo 2-propenyl)amino] 1-propanesulfonic acid, partially or totally salified, in the form of a salt of an alkali metal such as for example the sodium salt or the potassium salt, the ammonium salt, or a salt of an amino alcohol such as for example the salt of monethanolamine or a salt of an amino acid such as for example the lysine salt.

The weak acidic function of the monomer in question is particularly the carboxylic acid function, and preferably, said monomer is selected from acrylic acid, methacrylic acid, itaconic acid or maleic acid, partially or totally salified.

The neutral monomer is particularly selected from 2-hydroxy ethyl acrylate, 2,3-dihydroxy propyl acrylate, 2-hydroxy ethyl methacrylate, 2,3-dihydroxy propyl methacrylate, or an ethoxylated derivative of a molecular weight comprised between 400 and 1000, of each of these esters.

According to a fourth particular aspect of the present invention, the polyelectrolyte comprised in the inverse latex as defined above, is a homo-polymer of acrylic acid partially or totally salified, in the form of the sodium salt or the ammonium salt.

According to a fifth particular aspect of the present invention, the polyelectrolyte comprised in the inverse latex as described above, is a copolymer comprising by molar proportion between 30% and 90% and more particularly between 50% and 90% of 2-methyl 2-[(1-oxo 2-propenyl)amino] 1-propane-sulfonic acid, partially or totally salified, and between 10% and 70% and more particularly between 10% and 50% of 2-hydroxy ethyl acrylate. The inverse latex as defined above, comprises more particularly 20% by weight to 60% by weight and preferably 25 to 45% by weight of the copolymer as defined above. Preferably it is an inverse latex as defined above, a copolymer comprising in molar proportion from 60% to 90% of sodium salt or ammonium salt of 2-methyl 2-[1-oxo 2-propenyl)amino] 1-propanesulfonic acid and from 10% to 40% of 2-hydroxy ethyl acrylate.

According to a sixth particular aspect of the present invention, the polyelectrolyte comprised in the inverse latex as defined above, is a copolymer comprising in molar proportions between 30% and 90% and more particularly between 30 and 45% of the sodium salt or of ammonium salt, of the monoethanolamine salt or of the lysine salt of 2-methyl 2-[(1-oxo 2-propenyl)amino] 1-propanesulfonic acid and from 10% to 70% and more particularly from 55% to 75% of acrylic acid partially or totally salified, in the form of a sodium salt, the ammonium salt, the monoethanolamine salt or the lysine salt.

The invention more particularly has for its object a composition as described above, characterized in that the polyelectrolyte is reticulated and/or branched with a diethylene or polyethylene compound in the molar proportion expressed by the ratio to the monomers used, of 0.005% to 1%, more particularly 0.01% to 0.5% and, most particularly, 0.1% to 0.25%.

The reticulation and/or branching agent is selected from diallyloxyacetic acid or one of its salts, such as sodium diallyloxyacetate, ethyleneglycol dimethacrylate, ethyleneglycol diacrylate, diallyl urea, trimethylol propanetriacrylate, methylene-bis(acrylamide), triallylamine or a mixture of these compounds.

The inverse latex as described above, generally contains from 4% to 10% by weight, of emulsifying agents. Generally, from 20% to 50% and more, particularly from 25 to 40% by weight of the total of the emulsifiers are of the water in oil type and 80% to 50% and more particularly 75% to 60% are of the oil in water type.

Its oil phase represents from 15 to 40% and more preferably from 20 to 25% of its total weight. This latex can moreover contain one or several additives selected particularly from complexing agents, transfer agents or chain limiting agents.

The invention thus has for its object a cosmetic, dermopharmaceutical or pharmaceutical composition, characterized in that it comprises at least one thickening compound at least one inverse latex as defined above.

The cosmetic, dermocosmetic, dermopharmaceutical or pharmaceutical compound defined above comprises generally 0.1% to 10% and more particularly between 0.5% and 5% by weight of said inverse latex. It is particularly present in the form of a milk, a lotion, a gel, a cream, a soap, a foaming bath, a balm, a shampoo or an after shampoo.

Generally speaking, said inverse latex can desirably replace the product sold under the name SEPIGEL™ 305 or SEPIGEL™ 501 by the applicant, is cosmetic, dermopharmaceutical or pharmaceutical compositions, because it also has a good compatibility with the other excipients used for the preparation of formulations such as milks, lotions, creams, soaps, baths, balms, shampoo or after shampoo. It can also be used in combination with said SEPIGEL. It is particularly compatible with the concentrates described and claimed in the International publications WO 92/06778, WO 95/04592, WO95/13863, WO 98/47610 or FR 2 734 496 or with surface active agents described in WO 93/08204. It is particularly compatible with MONTANOV™ 68, MONTANOV™ 82, MONTANOV™ 202 and MONTANOV™ WO18, MONTANOV™ S or SEPIPERL™ N. It can also be used in emulsions of the type described and claimed in EP 0 629 396 and in the cosmetic or physiologically acceptable aqueous dispersions with an organo-polysiloxane compound selected for example from those described in WO 93/05762 or in WO 93/21316. It can also be used to form aqueous gels of acid pH that are cosmetically or physiologically acceptable, such as those described in WO 93/07856; it can also be used in association with non-ionic celluloses, to form for example hair gels, such as those described in EP 0 684 024 or else in association with fatty acid esters and sugar, to form compositions for the treatment of the hair or skin such as those described in EP 0 603 019, or else in shampoos or after shampoos such as described and claimed in WO 92/21316 or else in association with a anionic homopolymer such as CARBOPOL™ to form products for hair treatment such as those described in DE 195 23596. It is also compatible with numerous active principals, such as for example autobronzing agents such as dihydroxyacetone (DHA) or anti-acne agents; it can thus be introduced into auto-bronzing compositions such as those described in EP 0 715 845 or EP 0 604 249, EP 0 576 188 or WO 93/07902. It is also compatible with N-acylated amino acid derivatives, which permits its use in soothing compositions particularly for sensitive skin, such as those described or claimed in WO 92/21318, WO 94/27561 or WO 98/09611. It is also compatible with glycolic acids, with lactic acid, with salicylic acid retinoids, phenoxy ethanol, sugars, glyceraldehyde, xanthanes, fruit acids, and various polyols used in the production of cosmetic formulations.

The invention therefore also has for its object, the use of an inverse latex as defined above, to prepare a cosmetic, dermocosmetic, dermopharmaceutical or pharmaceutical composition.

The examples which follow have the aim of illustrating the present invention without however limiting it. They show that the new inverse latexes do not irritate the skin and that their physical properties permit their use in the preparation of cosmetic, dermopharmaceutical or pharmaceutical compositions, more particularly adapted for the treatment of sensitive skin.

A) EXAMPLES OF PREPARATION OF COMPOSITIONS ACCORDING TO THE INVENTION

### Inverse Latex of an AMPS Copolymer (Sodium Salt)/Acrylic Acid (Sodium Salt), Reticulated to Methylene Bis(Acrylamide), in MARCOL™ 52 (Composition 1)

a) There is loaded into a beaker with agitation:

61.4 grams glacial acrylic acid,

470.2 grams of a 55% commercial solution of sodium 2-methyl 2-[(1-oxo 2-propenyl)amino] 1-propanesulfonate,

35.54 g of an aqueous solution of 48% by weight of sodium hydroxide,

0.45 g of a 40% by weight aqueous solution of sodium diethylenetriaminepentacetate, and

0.128 g of methylene bis(acrylamide).

The pH of this aqueous solution is adjusted to 5.1 and there is added deionized water so as to bring the mass of the aqueous phase to 643.8 g.

b) An organic phase is prepared by mixing:

234.5 g of MARCOL™ 52,

41.4 g of MONTANE™ 80 VG and

0.20 g of azo bis(isobutyronitrile).

c) The aqueous phase is progressively introduced into the organic phase and the whole is strongly agitated with an ULTRA-TURRAX™ agitator sold by IKA. The obtained emulsion is then transferred to a polymerization reactor, subjected to bubbling with nitrogen and then cooled to about 5-6° C. There is then added 10 ml of a solution containing 0.28% by weight of cumene hydroperoxide in MARCOL™ 52, then after homogenization of the solution, an aqueous solution of sodium metabisulfite (2.5 g in 100 ml of water) at the rate of 0.5 ml/minute for about 60 minutes letting the temperature rise to the pulmerization temperature. The reaction mixture is then maintained for about 90 minutes at this temperature, at the end of which the obtained mixture is cooled to about 35°. There is slowly introduced 69.35 g of sorbitan oleate ethoxylated to 20 moles (MONTANOX™ 80) and there is obtained the desired water in oil emulsion.

Physical Properties

Viscosity in water at 3% of the latex (Brookfield RVT Mobile 6, speed 5):

η=52,000 mPas

Viscosity in water at 3% of the latex (Brookfield RVT Mobile 6, speed 20):

η=17,700 mPas.

### Example 2

### Inverse Latex of a Sodium Salt AMPS Copolymer/Sodium Salt Acrylic Acid, Reticulated with Methylene Bis(Acrylamide), in Hydrogenated Polyisobutene (Composition 2)

a) There is loaded into a beaker with agitation:

61.4 g of acrylic acid,

470.2 g of a commercial solution of 55% of sodium 2-methyl 2-[(1-oxo 2-propenyl)amino] 1-propanesulfonate,

35.54 g of an aqueous solution of 48% by weight of sodium hydroxide,

0.45 g of an aqueous solution of 40% by weight, of sodium diethylenetriaminepentacetate, and

0.128 g of methylene bis(acrylamide).

The pH of this aqueous solution is adjusted to 5.1 and there is added deionized water so as to bring the mass of the aqueous phase to 643.8 g.

b) An organic phase is prepared by mixing:

260 g of hydrogenated polyisobutene,

30.7 g of MONTANE™ 80 VG,

0.2 g of azo bis(isobutyronitrile).

c) The aqueous phase is introduced progressively into the organic phase and the whole is strongly agitated by means of an ULTRA-TURRAX™ agitator sold by IKA. The obtained emulsion is then transferred into a polymerization reactor, subjected to bubbling with nitrogen and then cooled to about 5-6° C. There is then added 5 ml of a solution containing 0.75% by weight of cumene hydroperoxyde in the hydrogenated polyisobutene, then, after homogenization of the solution, an aqueous solution of sodium metabisulfite (2.5 g in 100 ml of water) at the rate of 0.5 ml/minute for about 60 minutes and the temperature is permitted to rise to the polymerization temperature. The reaction medium is then maintained for about 90 minutes at this temperature, at the end of which the obtained mixture is cooled to about 35°. There is slowly introduced 50 g of sorbitan oleate ethoxylated to 20 moles (MONTANOX™ 80) and there is obtained the desired water in oil emulsion.

Physical Properties

Viscosity in water at 3% of the latex (Brookfield RVT Mobile 6, speed 5):

η=89,200 mPas

Viscosity at 3% of latex+in salt water (NaCl 0.1%) (Brookfield RVT Mobile 3, speed 5):

η=10,500 mPas.

### Example 3

### Inverse Latex of an AMPS Copolymer (Sodium Salts)/Acrylic Acid (Sodium Salt), Reticulated with Methylene Bis(Acrylamide), in Hydrogenated Polyisobutene (Composition 3)

a) There is loaded into a beaker with agitation:

61.4 g of acrylic acid,

470.2 g of a 55% commercial solution of sodium 2-methyl 2-[(1-oxo 2-propenyl)amino] 1-propanesulfonate,

35.54 g of an aqueous solution of 48% by weight of sodium hydroxide,

0.45 g of an aqueous solution at 40% by weight, of sodium diethylenetriaminepentaetate, and

0.128 g of methylene bis(acrylamide).

The pH of this aqueous solution is adjusted to 5.1 and there is added the ionized water so as to bring the mass of the aqueous phase to 643.8 g.

b) An organic phase is prepared by mixing:

260 g of hydrogenated polyisobutene,

30.7 g of MONTANE™ 80 VG,

0.2 g of azo bis(isobutyronitrile).

c) The aqueous phase is progressively introduced into the organic phase and the whole is strongly agitated by means of an ULTRA-TURRAX™ agitator sold by IKA. The obtained emulsion is then transferred into a pulmerization reactor, subjected to bubbling with nitrogen and then cooled to about 5-6° C. There is then added 5 ml of a solution containing 0.75% by weight of cumene hydroperoxide in hydrogenated polyisobutene, then, after homogenization of the solution, an aqueous solution of sodium metabisulfite (2.5 g in 100 ml of water) at a rate of 0.5 ml/minute for about 60 minutes while letting the temperature rise to the polymerization temperature. The reaction medium is then maintained for about 90 minutes at this temperature, at the end of which the obtained mixture is cooled to about 35°. There is slowly introduced 54.5 g of SIMULSOL™ SL8 and there is obtained the desired water in oil emulsion.

Physical Properties

Viscosity in water at 3% of the latex (Brookfield RVT Mobile 6, speed 5):

η=87,600 mPas

Viscosity at 3% of the latex+in salt water (NaCl 0.1%) (Brookfield RVT Mobile 3, speed 5):

η=16,800 mPas.

### Example 4

### Inverse Latex of a Sodium Salt of AMPS/Sodium Salt of Acrylic Acid, Reticulated with Methylene Bis(Acrylamide), in Hydrogenated Polyisobutene (Composition 4)

a) There is loaded into a beaker with agitation:

61.4 g of acrylic acid,

470.2 g of a commercial solution of 55% of sodium 2-methyl 2-[(1-oxo 2-propenyl)amino] 1-propanesulfonate,

35.54 g of an aqueous solution of 48% by weight of sodium hydroxide,

0.45 g of an aqueous solution of 40% by weight of sodium diethylenetriaminepentacetate, and

0.128 g of methylene bis(acrylamide).

The pH of this aqueous solution is adjusted to 5.1 and there is added deionized water so as to bring the mass of the aqueous phase to 643.8 g.

b) An organic phase is produced by mixing:

260 g of hydrogenated polyisobutene,

30.7 g of MONTANE™ 80 VG,

0.2 g of azo bis(isobutyronitrile).

c) The aqueous phase is introduced progressively into the organic phase and the whole is agitated strongly by means of an ULTRA-TURRAX™ agitator sold by IKA. The obtained emulsion is then transferred into a polymerization reactor, subjected to bubbling with nitrogen and then cooled to about 5-6° C. There is then added 5 ml of a solution containing 0.75% by weight of cumene hydroperoxide in the hydrogenated polyisobutene, then after homogenization of the solution, an aqueous solution of sodium metabisulfite (2.5 g in 100 ml of water) at a rate of 0.5 ml/minute for about 60 minutes while letting the temperature rise to the polymerization temperature. The reaction medium is then maintained for about 90 minutes at this temperature, at the end of which the obtained mixture is cooled to about 35° C. There is slowly introduced 72.7 g of SIMULSOL™ SL26 and there is obtained the desired water in oil emulsion.

Physical Properties

Viscosity in water at 3% of the latex (Brookfield RVT Mobile 6, speed 5):

η=79,600 mPas

Viscosity at 3% of the latex+in salt water (0.1% sodium chloride) (Brookfield RVT Mobile 3, speed 5):

η=10,600 mPas.

### Example 5

### Inverse Latex of an AMPS Copolymer (Lysine Salt)/Acrylic Acid (Lysine Salt), Reticulated with Methylene Bis(Acrylamide), in Squalene (Composition 5)

a) There is loaded into a beaker with agitation:

61.4 g of acrylic acid,

470.2 g of a commercial solution of 55% sodium 2-methyl 2-[(1-oxo 2-propenyl)amino] 1-propanesulfonate,

0.46 g of an aqueous solution of 40% by weight of sodium diethylenetriaminepentacetate,

0.161 g of methylene bis(acrylamide),

70.0 g of L-Lysine and

76.13 g of water.

The quantity of L-Lysine is adjusted to obtain a pH of the solution near 5.0. The speed of addition is such that the temperature of the reaction medium does not exceed 25° C.

b) There is prepared an organic phase by mixing:

234.5 g of squalene,

41.4 g of MONTANE™ 80 VG,

0.2 g of azo bis(isobutyronitrile).

c) The aqueous phase is introduced progressively into the organic phase and the whole is agitated strongly with an ULTRA-TURRAX™ mixer sold by IKA. The obtained emulsion is then transferred into a polymerization reactor, subjected to bubbling with nitrogen then cooled to about 5-6° C. There is then added 10 ml of a solution containing 0.28% by weight of cumene hydroperoxide in the squalene, then, after homogenization of the solution, an aqueous solution of sodium metabisulfite (2.5 g in 100 ml of water) at a rate of 0.5 ml/minute for about 60 minutes and letting the temperature rise to 75° C. The reaction medium is then maintained for about 60 minutes at this temperature, at the end of which the obtained mixture is cooled to about 35° C. There is slowly introduced 50 g of MONTANOX™ 80 and there is obtained the desired water in oil emulsion.

Physical Properties

Viscosity in water at 3% of the latex (Brookfield RVT Mobile 6, speed 5):

η=51,000 mPas

Viscosity at 3% of the latex in an aqueous salt solution (0.1% NaCl) (Brookfield RVT Mobile 3, speed 5):

η=6,700 mPas

pH of the aqueous solution at 3% latex: 6.3

### Example 6

### Inverse Latex of an AMPS Copolymer (Monoethanolamine Salt)/Acrylic Acid (Monoethanolamine Salt), Reticulated with Methylene Bis(Acrylamide), in Squalene (Composition 6)

a) There is loaded into a beaker with agitation:

61.4 g of acrylic acid,

470.2 g of a commercial solution of 55% of sodium 2-methyl 2-[(1-oxo 2-propenyl)amino] 1-propanesulfonate,

0.46 g of an aqueous solution of 40% by weight of sodium diethylenetriaminepentacetate, and

0.161 g of methylene bis(acrylamide),

26.0 g of monoethanolamine and

90.4 g of water.

b) An organic phase is prepared by mixing:

234.5 g of squalene,

41.4 g of MONTANE™ 80 VG,

0.2 g of azo bis(isobutyronitrile).

c) The aqueous phase is introduced progressively into the organic phase and the whole is agitated strongly with an ULTRA-TURRAX™ agitator sold by IKA. The obtained emulsion is then transferred to a polymerization reactor, subjected to bubbling with nitrogen and then cooled to about 5-6° C. There is then added 10 ml of a solution containing 0.28% by weight of cumene hydroperoxide in the squalene then, after homogenization of the solution, an aqueous solution of sodium metabisulfite (2.5 g in 100 ml of water) at a rate of 0.5 ml/minute for about 60 minutes while letting the temperature rise to 75° C. The reaction medium is thus maintained for about 60 minutes at this temperature, at the end of which the obtained mixture is cooled to about 35° C. There is slowly introduced 50 g of MONTANOX™ 80 and there is obtained the desired water in oil emulsion.

Physical Properties

Viscosity in water at 3% of the latex (Brookfield RVT Mobile 6, speed 5):

η=69,200 mPas

Viscosity at 3% of the latex in an aqueous salt solution (0.1% NaCl) (Brookfield RVT Mobile 3, speed 5):

η=6,300 mPas

pH of the aqueous solution at 3% of the latex: 6.0

### Example 7

### Inverse Latex of an AMPS Copolymer (Sodium Salt)/2-hydroxy Ethyl Acrylate, Reticulated with Methylene Bis(acrylamide), in Squalene (Composition 7)

a) There is loaded in a beaker with agitation:

20.4 g of (2-hydroxy ethyl) acrylate,

660.6 g of a commercial solution of 55% by weight of sodium 2-methyl 2-[(1-oxo 2-propenyl)amino] 1-propanesulfonate,

0.45 g of an aqueous solution of 40% by weight, of sodium diethylenetriaminepentacetate, and

0.123 g of methylene bis(acrylamide).

The pH is adjusted to 4.0 by adding 0.55 g of 2-methyl 2-[(1-oxo 2-propenyl)amino] 1-propanesulfonic acid.

b) An organic phase is prepared by mixing:

265 g of squalene,

17.76 g of MONTANE™ 80 VG,

9.24 g of MONTANOX™ 81 VG,

0.2 g of azo bis(isobutyronitrile).

c) The aqueous phase is introduced progressively into the organic phase and the whole is agitated strongly by means of an ULTRA-TURRAX™ agitator sold by IKA. The obtained emulsion is then transferred into a polymerization reactor, subjected to bubbling with nitrogen and then cooled to about 5-6° C. There is then added 10 ml of a solution containing 0.28% by weight of cumene hydroperoxide into the squalene, then, after homogenization of the solution, an aqueous solution of sodium metabisulfite (2.5 g in 100 ml of water) at a rate of 0.5 ml/minute for about 60 minutes and letting the temperature rise to 75° C. The reaction medium is then maintained for about 60 minutes at this temperature, at the end of which the obtained mixture is cooled to about 35° C. There is slowly introduced 25 g of MONTANOX™ 80 and there is obtained the desired water in oil emulsion.

Physical Properties

Viscosity in water at 3% of the latex (Brookfield RVT Mobile 6, speed 5):

η=90,000 mPas

Viscosity at 3% of the latex in an aqueous salt solution (0.1% NaCl) at pH=3 (Brookfield RVT Mobile 6, speed 5):

η=58,800 mPas

pH of the aqueous solution at 3% of the latex: 5.3

### Example 8

### Inverse Latex of an AMPS Copolymer (Sodium Salt)/2-hydroxy Ethyl Acrylate, Reticulated with Methylene Bis(acrylamide), in Squalene (Composition 8)

a) There is loaded into a beaker with agitation:

20.4 g of (2-hydroxy ethyl) acrylate,

660.6 g of a commercial solution of 55% by weight of sodium 2-methyl 2-[(1-oxo 2-propenyl)amino] 1-propanesulfonate,

0.45 g of an aqueous solution of 40% by weight, of sodium diethylenetriaminepentacetate, and

0.123 g of methylene bis(acrylamide).

The pH is adjusted to 4.0 by adding 0.55 g of 2-methyl 2-[(1-oxo 2-propenyl)amino] 1-propanesulfonic acid.

b) An organic phase is prepared by mixing:

265 g of squalene,

17.76 g of MONTANE™ 80 VG,

9.24 g of MONTANOX™ 81 VG,

0.2 g of azo bis(isobutyronitrile).

c) The aqueous phase is introduced progressively into the organic phase and the whole is agitated strongly by means of an ULTRA-TURRAX™ agitator sold by IKA. The obtained emulsion is then transferred into a polymerization reactor, subjected to bubbling with nitrogen and then cooled to about 5-6° C. There is then added 10 ml of a solution containing 0.28% by weight of cumene hydroperoxide into the squalene and then, after homogenization of the solution, an aqueous solution of sodium metabisulfite (2.5 g in 100 ml of water) at a rate of 0.5 ml/minute for about 60 minutes while letting the temperature rise to 75° C. The reaction mixture is then held for about 60 minutes at this temperature, at the end of which the obtained mixture is cooled to about 35° C. There is slowly introduced 27.2 g of SIMUSOL™ SL10 and there is obtained the desired water in oil emulsion.

Physical Properties

Viscosity in water at 3% of the latex (Brookfield RVT Mobile 6, speed 5):

η=113,000 mPas

Viscosity at 3% of the latex in an aqueous salt solution (0.1% NaCl) (Brookfield RVT Mobile 3, speed 5):

η=17,200 mPas

pH of the aqueous solution at 3% latex: 5.8

### Example 9

### Inverse Latex of an AMPS Copolymer (Sodium Salt)/2-Hydroxy Ethyl Acrylate, Reticulated with Methylene Bis(Acrylamide), in Squalene (Composition 9)

a) There is loaded into a beaker with agitation:

20.4 g of (2-hydroxy ethyl) acrylate,

660.6 g of a commercial solution of 55% of sodium 2-methyl 2-[(1-oxo 2-propenyl)amino] 1-propanesulfonate,

0.45 g of an aqueous solution of 40% by weight of sodium diethylenetriaminepentacetate, and

0.123 g of methylene bis(acrylamide).

The pH is adjusted to 4.0 by adding 0.55 g of 2-methyl 2-[(1-oxo 2-propenyl)amino] 1-propanesulfonic acid.

b) An organic phase is prepared by mixing:

265 g of squalene,

17.76 g of MONTANE™ 80 VG,

9.24 g of MONTANOX™ 81 VG,

0.2 g of azo bis(isobutyronitrile).

c) The aqueous phase is introduced progressively into the organic phase and the whole is agitated strongly by means of an ULTRA-TURRAX™ agitator sold by IKA. The obtained emulsion is then transferred into a polymerization reactor, subjected to bubbling with nitrogen and then cooled to about 5-6° C. There is then added 10 ml of a solution containing 0.28% by weight of cumene hydroperoxide into the squalene and then, after homogenization of the solution, an aqueous solution of sodium metabisulfite (2.5 g in 100 ml of water) at a rate of 0.5 ml/minute for about 60 minutes while letting the temperature rise to 75° C. The reaction medium is then held for about 60 minutes at this temperature, at the end of which the obtained mixture is cooled to about 35° C. There is slowly introduced 27.2 g of SIMUSOL™ SL8 and there is obtained the desired water in oil emulsion.

Physical Properties

Viscosity in water at 3% of the latex (Brookfield RVT Mobile 6, speed 5):

η=93,000 mPas

Viscosity at 3% of the latex in an aqueous salt solution (0.1% NaCl) (Brookfield RVT Mobile 6, speed 5):

η=6,100 mPas

pH of the aqueous solution at 3% latex: 5.8

B PROPERTIES OF THE COMPOSITIONS ACCORDING TO THE INVENTION

a) Temperature Stability

There is prepared a cream gel comprising 3% of composition 7 and 20% of cetearyl octanoate and the viscosity is measured. The results are as follows:

Brookfield viscosity LVT 6 rpm (in mPas)

| | At ambient temperature | At 50° C. |
|---|---|---|
| After 1 day | 90,000 mPas | 90,000 mPas |
| After 7 days | 90,000 mPas | 90,000 mPas |
| After 1 month | 90,000 mPas | 90,000 mPas |

b) Stability Under UV Radiation

It has been seen that the gel prepared with Composition 7 is very UV stable, because its viscosity has not varied after 14 days exposure.

c) Influence of pH on Viscosity

The viscosity of the cream gel prepared with Composition 7 is very stable as to pH over the pH range pH=3 to pH=8

d) Comparative Study of Cutaneous Tolerance

The local epicutaneous tolerance of a series of cream gels, containing 3% and 5% by weight of one of the Compositions 1 to 9 prepared as described above, has been determined and compared to that observed with an inverse latex of an AMPS copolymer/acrylate reticulated with methylene bis(acrylamide), in isohexadecane (Composition A), according to the following protocol:

The composition to be tested is applied to a surface of about 50 mm² of the left sub-capillary region, of the skin of the back of 38 healthy volunteers, among which 19 have a “Japanese skin” type (JS) and 19 have a “caucasian skin” type (CS). The contact is maintained for 48 hours under a sealing patch.

This application is also carried out under the same conditions with a single patch (without composition) as a negative blank.

Clinical observation of the surface of the skin thus treated is carried out 30 minutes and then 24 hours after applying said patches. These observations are made by comparison to the surface of the negative untreated blank.

The quantification of the cutaneous irritation, according to a numerical scale running from 0 to 4 (0: no effect; 1: very slight effect; 2: discernible effect; 3 and 4: moderate to severe effect according to the reactions), is carried out for each of the observed reactions, namely: erythema, edema, vesicles, dryness of the skin, roughness of the skin and reflectivity of the skin.

The indices of cutaneous tolerance (CI), expressing the mean of the sum of the quantified effects from each volunteer:

CI=0 means no irritation was observed,

CI≦0.5 means that the product is statistically well tolerated,

CI>0.5 means that the product gives rise to intolerance.

The results expressed in indices of cutaneous tolerance, are set forth in the following table:

| Index of cutaneous tolerance | |
|---|---|
| gel at 3% | gel at 5% |

| | J.S. | C.S. | J.S. | C.S. |
|---|---|---|---|---|
| Composition 1 | 0.26 | 0.00 | 0.26 | 0.05 |
| Composition 2 | n.a. | 0.00 | n.a. | 0.00 |
| Composition 3 | 0.89 | 0.21 | 0.63 | 0.05 |
| Composition 4 | 0.89 | 0.21 | 0.63 | 0.05 |
| Composition 5 | 1.00 | 0.05 | 1.11 | 0.05 |
| Composition 6 | n.a. | n.a. | n.a. | n.a. |
| Composition 7 | 1.00 | 0.00 | 1.11 | 0.05 |
| Composition 8 | n.a. | 0.1 | n.a. | 0.00 |
| Composition 9 | 0.53 | 0.0 | 0.95 | 0.00 |
| Composition A | 1.00 | 0.47 | 0.95 | 0.47 |

These results show that in a surprising manner, squalene, hydrogenated polyisobutene and MARCOL™ 52 potentialize the polymeric cutaneous tolerance of the inverse latex.

C) EXAMPLES OF FORMULATIONS PREPARED WITH COMPOSITIONS ACCORDING TO THE INVENTION

Example 10: Skin cream

| | |
|---|---|
| Cyclomethicone: | 10% |
| Composition 4: | 0.8% |
| MONTANOV ™ 68: | 2% |
| stearyl alcohol: | 1% |
| stearic alcohol: | 0.5% |
| preservative: | 0.65% |
| Lysine: | 0.025% |
| EDTA (disodium salt): | 0.05% |
| Xanthane gum: | 0.2% |
| Glycerine: | 3% |
| Water: | q.s.p. 100% |

Example 11: After shave balm
FORMULA

| | | |
|---|---|---|
| A | Composition 4: | 1.5% |
| | Water: | q.s.p. 100% |
| B | MICROPEARL ™ M 100: | 5.0% |
| | SEPICIDE ™ CI: | 0.50% |
| | Perfume: | 0.20% |
| | ethanol 95%: | 10.0% |

OPERATIVE MODE
Add B into A.

Example 12: Satinized body emulsion formula
FORMULA

| | | |
|---|---|---|
| A | SIMULSOL ™ 165: | 5.0% |
| | LANOL ™ 1688: | 8.50% |
| | Karita butter: | 2% |
| | paraffin oil: | 6.5% |
| | LANOL ™ 14M: | 3% |
| | LANOL ™ S: | 0.6% |
| B | water: | 66.2% |
| C | MICROPEARL ™ M 100: | 5% |
| D | Composition 3: | 3% |
| E | SEPICIDE ™ CI: | 0.3% |
| | SEPICIDE ™ HB: | 0.5% |
| | MONTEINE ™ CA: | 1% |
| | Perfume: | 0.20% |
| | vitamin E acetate: | 0.20% |
| | Sodium pyrolidinonecar- | 1% |
| | boxylate: | |

OPERATIVE MODE

Add C into B, emulsion B into A at 70° C., then
add D to C at 60° C., then E at 30° C.

Example 13: O/W Cream
FORMULA

| | | |
|---|---|---|
| A | SIMULSOL ™ 165: | 5.0% |
| | LANOL ™ 1688: | 20.0% |
| | LANOL ™ P: | 1.0% |
| B | water: | q.s.p. 100% |
| C | Composition 3: | 2.50% |
| D | SEPICIDE ™ CI: | 0.20% |
| | SEPICIDE ™ HB: | 0.30% |

OPERATIVE MODE

Introduce B into A at 75° C.; introduce C
at 60°, then D at 45° C.

Example 14: Non-greasy sun gel
FORMULA

| | | |
|---|---|---|
| A | Composition 5: | 3.00% |
| | Water: | 30% |
| B | SEPICIDE ™ CI: | 0.20% |
| | SEPICIDE ™ HB: | 0.30% |
| | Perfume: | 0.10% |
| C | colorant: | q.s.p. |
| | water: | 30% |
| D | MICROPEARL ™ M 100: | 3.00% |
| | Water: | q.s.p. 100% |
| E | silicone oil: | 2.0% |
| | PARSOL ™ MCX: | 5.00% |

OPERATIVE MODE
Introduce B into A; add C, then D, then E.

Example 15: Solar milk
FORMULA

| | | |
|---|---|---|
| A | MONTANOV ™ S: | 3.0% |
| | sesame oil: | 5.0% |
| | PARSOL ™ MCX: | 5.0% |
| | Carraghenan λ: | 0.10% |
| B | water: | q.s.p. 100% |
| C | Composition 1: | 0.80% |
| D | Perfume: | q.s. |
| | Preservative: | q.s. |

OPERATIVE MODE

Emulsionize B in A at 75° C. then add C at 60 ° C.,
then D at 30° C. and adjust the pH if necessary

Example 16: Message gel
FORMULA

| | | |
|---|---|---|
| A | Composition 2: | 3.5% |
| | Water: | 20.0% |
| B | colorant: | 2 drops/100 g |
| | Water: | q.s. |
| C | alcohol: | 10% |
| | Menthol: | 0.10% |
| D | silicone oil: | 5.0% |

OPERATIVE MODE
Add B into A, then add to the mixture C then D

Example 17: Hydrating and matifying color base
FORMULA

| | | |
|---|---|---|
| A | water: | 20.0% |
| | Butylene glycol: | 4.0% |
| | PEG-400: | 4.0% |
| | PECOSIL ™ PS100: | 1.0% |
| | NaOH: | q.s. pH = 9 |
| | Titanium dioxide: | 7.0% |
| | Talc: | 2.0% |
| | Yellow iron oxide: | 0.8% |
| | Red iron oxide: | 0.3% |
| | Black iron oxide: | 0.05% |
| B | LANOL ™ 99: | 8% |
| | Caprylic capric triglyceride | 8% |
| | MONTANOV ™ 202: | 5.00% |
| C | water: | q.s.p. 100% |
| | MICROPEARL ™ M305: | 2.0% |
| | EDTA tetrasode: | 0.05% |
| D | Cyclomethicone: | 4.0% |
| | Xanthan gum: | 0.2% |
| | composition 7: | 0.8% |
| E | SEPICIDE ™ HB: | 0.5% |
| | SEPICIDE CI: | 03% |
| | Perfume: | 0.2% |

OPERATIVE MODE

prepare at 80° C.,
mixtures B + D and A + C,
then mix and emulsify the whole.

Example 18: Super gel
FORMULA

| | | |
|---|---|---|
| A | Composition 6: | 4% |
| | Water: | 30% |
| B | ELASTINE HPM: | 5.0% |
| C | MICROPEAL ™ M 100: | 3% |
| | Water: | 5% |
| D | SEPICIDE ™ CI: | 0.2% |
| | SEPICIDE ™ HB: | 0.3% |
| | Perfume: | 0.06% |
| | Sodium pyrolidinone- | 1% |
| | carboxylate 50%: | |
| | Water: | q.s.p. 100% |

OPERATIVE MODE
Prepare A; add B, then C, then D.

Example 19: Body milk
FORMULA

| | |
|---|---|
| MONTANOV ™ S: | 3.5% |
| LANOL ™ 37T″ | 8.0% |
| SOLAGUM ™ L: | 0.05% |
| Water: | q.s.p. 100% |
| Benzophenone: | 2.0% |
| dimethicone 350cPs: | 0.05% |
| Composition 5: | 0.8% |
| preservative: | 0.2% |
| perfume: | 0.4% |

Example 20: Makeup cleansing emulsion with sweet almond oil
FORMULA

| | |
|---|---|
| MONTANOV ™ 68: | 5% |
| sweet almond oil: | 5% |
| water: | q.s.p. 100% |
| Composition 9: | 0.3% |
| glycerine: | 5% |
| preservative: | 0.2% |
| perfume: | 0.3% |

Example 21: Hydrating cream for oily skin
FORMULA

| | |
|---|---|
| MONTANOV ™ 68: | 5% |
| Cetylstearyloctanoate: | 8% |
| octyl palmitate: | 2% |
| water: | q.s.p. 100% |
| Composition 5: | 0.6% |
| MICROPEARL ™ M100: | 3.0% |
| Mucopolysaccharides: | 5% |
| SEPICIDE ™ HB: | 0.8% |
| Perfume: | 0.3% |

Example 22: Soothing after-shave balm without alcohol
FORMULA

| | | |
|---|---|---|
| A | LIPACIDE ™ PVB: | 1.0% |
| | LANOL ™ 99: | 2.0% |
| | Sweet almond oil: | 0.5% |
| B | Composition B: | 3.5% |
| C | water: | q.s.p. 100% |
| D | perfume: | 0.4% |
| | SEPICIDE ™ HB: | 0.4% |
| | SEPICIDE ™ CI: | 0.2% |

Example 23: Cream with AHA for sensitive skin
FORMULA

| | |
|---|---|
| Mixture of lauryl | 0.1% |
| amino acids: | to 5% |
| Magnesium and potassium | 0.002% |
| aspartate: | to 0.5% |
| LANOL ™ 99: | 2% |
| MONTANOV ™ 68: | 5.0% |
| Water: | q.s.p. 100% |
| Composition 7: | 1.50% |
| gluconic acid: | 1.50% |
| tri ethylamine: | 0.9% |
| SEPICIDE ™ HB: | 0.3% |
| SEPICIDE ™ CI: | 0.2% |
| Perfume: | 0.4% |

Example 24: Soothing after sun care
FORMULA

| | |
|---|---|
| mixture of lauryl amino | 0.1% |
| acids: | to 5% |
| magnesium and potassium | 0.002% |
| aspartate | to 0.5% |
| LANOL ™ 99: | 10.0% |
| Water: | q.s.p. 100% |
| Composition 1: | 2.50% |
| SEPICIDE ™ HB: | 0.3% |
| SEPICIDE ™ CI: | 0.2% |
| Perfume: | 0.4% |
| Colorant: | 0.03% |

Example 25: Makeup removing milk
FORMULA

| | |
|---|---|
| SEPIPERL ™ N: | 3% |
| PRIMOL ™ 352: | 8.0% |
| sweet almond oil: | 2% |
| water: | q.s.p. 100% |
| Composition 7: | 0.8% |
| preservative: | 0.2% |

Example 26: Fluid emulsion with alkaline pH

| | |
|---|---|
| MARCOL ™ 82: | 5.0% |
| NaOH: | 10.0% |
| Water: | q.s.p. 100% |
| Composition 8: | 1.5% |

Example 27: Fluid color base
FORMULA

| | |
|---|---|
| SIMULSOL ™ 165: | 5.0% |
| LANOL ™ 84D | 8.0% |
| LANOL ™ 99: | 5.0% |
| Water: | q.s.p. 100% |
| pigments and mineral fillers: | 10.0% |
| Composition 2: | 1.2% |
| preservative: | 0.2% |
| perfume: | 0.4% |

Example 28: Sun milk
FORMULA

| | |
|---|---|
| SEPIPERL ™ N: | 3.5% |
| LANOL ™ 37T: | 10.0% |
| PARSOL ™ MCX: | 5.0% |
| EUSOLEX ™ 4360: | 2.0% |
| Water: | q.s.p. 100% |
| Composition 6: | 1.8% |
| preservative: | 0.2% |
| perfume: | 0.4% |

Example 29: Eye-outlining gel
FORMULA

| | |
|---|---|
| Composition 7: | 2.0% |
| Perfume: | 0.06% |
| Sodium | 0.2% |
| pyrrolidinonecarboxylate: | |
| DOW CORNING ™ 245 Fluid: | 2.0% |
| Water: | q.s.p. 100% |

Example 30: Composition of unrinsed solution
FORMULA

| | |
|---|---|
| Composition 5: | 1.5% |
| Perfume: | q.s. |
| Preservative: | q.s. |
| DOW CORNING ™ X2 8360: | 5.0% |
| DOW CORNING ™ Q2 1401: | 15.0% |
| Water: | q.s.p. 100% |

Example 31: Thinning gel

| | |
|---|---|
| Composition 7: | 5% |
| Ethanol: | 30% |
| Menthol: | 0.1% |
| Caffeine: | 2.5% |
| extract of ruscus: | 2% |
| extract of ivy: | 2% |
| SEPICIDE ™ HP: | 1% |
| Water: | q.s.p. 100% |

Example 32: Ultra natural colored gel cream

| FORMULA | | |
|---|---|---|
| A | water: | 10.0% |
| | Butylene glycol: | 4.0% |
| | PEG-400: | 4.0% |
| | PECOSIL ™ PS100: | 1.5% |
| | NaOH: | q.s. pH = 7 |
| | Titanium dioxide: | 2.0% |
| | Yellow iron oxide: | 0.8% |
| | Red iron oxide: | 0.3% |
| | Black iron oxide: | 0.05% |
| B | LANOL ™ 99: | 4.0% |
| | Caprylic capric | 4.0% |
| | triglyceride: | |
| | SEPIFEEL ™ ONE: | 1.0% |
| | composition 9: | 3.0% |
| C | water: | q.s.p. 100% |
| | MICROPEARL ™ M305: | 2.0% |
| | tetrasodium EDTA: | 0.05% |
| | Cyclomethicone: | 4.0% |
| D | SEPICIDE ™ HB: | 0.5% |
| | SEPICIDE CI: | 03% |
| | Perfume: | 0.2% |

OPERATIVE MODE
preparing mixture B + C then A and then D.

Example 33: Oily skin care
FORMULA

| | | |
|---|---|---|
| A | MICROPEARL ™ M310: | 1.0% |
| | Composition 5: | 5.0% |
| | Octyl isononanoate | 4.0% |
| B | water: | q.s.p. 100% |
| C | SEPICONTROL ™ AS: | 4.0% |
| | Perfume: | 0.1% |
| | SEPICIDE ™ HB: | 0.3% |
| | SEPICIDE ™ CI: | 0.2% |
| D | CAPIGEL ™ 98: | 0.5% |
| | Water: | 10% |

Example 34: AHA Cream
FORMULA

| | | |
|---|---|---|
| A | MONTANOV ™ 68: | 5.0% |
| | LIPACIDE ™ PVB: | 1.05% |
| | LANOL ™ 99: | 10.0% |
| B | water: | q.s.p. 100% |
| | Gluconic acid: | 1.5% |
| | Triethanolamine: | 0.9% |
| C | Composition 2: | 1.5% |
| D | perfume: | 0.4% |
| | SEPICIDE ™ HB: | 0.2% |
| | SEPICIDE ™ CI: | 0.4% |

Example 35:
Non-greasy auto-bronzing agent for the face and body
FORMULA

| | | |
|---|---|---|
| A | LANOL ™ 2681: | 3.0% |
| | Composition 1: | 2.5% |
| B | water: | q.s.p. 100% |
| | Dihydroxyacetone: | 3.0% |
| C | perfume: | 0.2% |
| | SEPICIDE ™ HB: | 0.8% |
| | NaOH (sodium hydroxide): | q.s. pH = 5 |

Example 36:
Sun milk with Tahiti Monoi
FORMULA

| | | |
|---|---|---|
| A | Tahiti Monoi: | 10% |
| | LIPACIDE ™ PVB: | 0.5% |
| | Composition 2: | 2.2% |
| B | water: | q.s.p. 100% |
| C | perfume: | 0.1% |
| | SEPICIDE ™ HB: | 0.3% |
| | SEPICIDE ™ CI: | 0.1% |
| | PARSOL ™ MCX: | 4.0% |

Example 37: Sun care for the face
FORMULA

| | | |
|---|---|---|
| A | Cyclomethicone and | 4.0% |
| | dimethiconol: | |
| | Composition 3: | 3.5% |
| B | water: | q.s.p. 100% |
| C | perfume: | 0.1% |
| | SEPICIDE ™ HB: | 0.3% |
| | SEPICIDE ™ CI: | 0.21% |
| | PARSOL ™ MCX: | 5.0% |
| | Micatitania: | 2.0% |
| | Lactic acid: | q.s.p. |
| | | pH = 6.5 |

Example 38: Sunless bronzing emulsion
FORMULA

| | | |
|---|---|---|
| A | LANOL ™ 99: | 15% |
| | MONTANOV ™ 68: | 5.0% |
| | PARSOL ™ MCX: | 3.0% |
| B | water: | q.s.p. 100% |
| | Dihydroxyacetone: | 5.0% |
| | Monosodium phosphate: | 0.2% |
| C | Composition 4: | 0.5% |
| D | perfume: | 0.3% |
| | SEPICIDE ™ HB: | 0.8% |
| | NaOH: | q.s. |
| | | pH = 5. |

The characteristics of the products used in the preceding examples, are as follows:

MONTANOV™ 68 (cetearyl glucoside, cetearylic alcohol), is a self-emulsionable composition such as those described in WO 92/06778, sold by the SEPPIC company.

MONTANOV™ 202 (arachidyl glucoside, arachydilic alcohol+behenylic alcohol), is a self-emulsionable composition such as those described in WO 98/17610, sold by the SEPPIC company.

MICROPEARL™ M 305 is a hydrodispersible soy powder based on reticulated methylmethacrylate copolymer.

MICROPEARL™ M 100 is a powder that is ultrafine to the touch, very soft and with a matifying action, sold by the MATSUMO company.

SEPICIDE™ CI, ureic imidazoline, is a preservative agent sold by the SEPPIC company.

PREMULEN™ TR is an acrylic polymer sold by GOODRICH.

SIMULSOL™ 165 is a self-emulsionable glycerol stearate sold by the SEPPIC company.

LANOL™ 1688 is an emollient ester with a non-greasy effect, sold by the SEPPIC company.

LANOL™ 14M and LANOL™ S are consistency factors sold by the SEPPIC company.

SEPICIDE ™ HB, which is a mixture of phenoxyethanol, methylparaben, ethylparaben, propylparaben and butylbaraben, is a preservative agent sold by the SEPPIC company.

MONTEINE™ CA is a hydrating agent sold by the SEPPIC company.

SCHERCEMOL™ OP is an emollient ester with a non-greasy effect.

LANOL™ P is an additive with a stabilizing effect sold by the SEPPIC company.

SEPIPERL™ N is a nacrifying agent sold by the SEPPIC company, with a base of a mixture of alkyl polyglucosides such as those described in WO 95/13863.

MONTANOV™ S is a nacrifying agent, sold by the SEPPIC company, based on a mixture of alkyl polyglucosides such as those described in WO 95/13863.

PECOSIL™ PS100 is dimethicone copolyol phosphate sold by the PHOENIX company.

LANOL™ 99 is isononyl isononanoate sold by the SEPPIC company.

LANOL™ 37T is a glycerol triheptanoate, sold by the SEPPIC company.

SEPIFEEL™ ONE is a mixture of palmitoylproline, magnesium palmitoyl glutamate and magnesium palmitoyl sarcosinate, such as described in FR 2787323.

SOLAGUM™ L is a carraghenate sold by the SEPPIC company.

MARCOL™ 82 is a paraffin oil sold by the ESSO company.

LANOL™ 84D is dioctyl malate sold by the SEPPIC company.

PARSOL™ MCX is ethyl hexyle paramethoxycinnamate, sold by the GIVAUDAN company.

EUSOLEX™ 4360 is benzophenone-3 sold by the MERCK company.

DOW CORNING™ 245 Fluid is cyclomethicone sold by DOW CORNING.

LIPACIDE™ PVB is a palmitoylated hydrolysate of wheat proteins, sold by the SEPPIC company.

SEPICONTROL™ A5 is a mixture of capryloyl glycine, sarcosine, extract of cinnamon zylanicum, sold by the SEPPIC company, as described in International patent application PCT/FR98/01313 filed Jun. 23, 1998.

CAPIGEL™ 98 is an acrylate copolymer sold by the SEPPIC company.

LANOL™ 2681 is a mixture of caprylate, copra caprate, sold by the SEPPIC company.

1. Composition comprising an oil phase, an aqueous phase, at least one emulsifying agent of the water in oil (W/O) type, at least one emulsifying agent of the oil in water (O/W) type, in the form of an auto-inversible inverse latex comprising from 20% to 70% by weight, and preferably from 25% to 50% by weight, of a branched or reticulated polyelectrolyte, characterized in that said polyelectrolyte is either a homopolymer based on a monomer having a weakly acidic function partially or totally salified, or a copolymer with a base of at least one monomer having a strongly acidic function, copolymerized either with at least one monomer having a weakly acidic function, or with at least one neutral monomer, and characterized in that the constituent solvent of the oil phase is selected from white mineral oil, squalene or hydrogenated polyisobutene.

2. Composition as defined in claim 1, in which the solvent constituting the oil phase is a white mineral oil and is more particularly MARCOL™ 52.

3. Composition as defined in claim 1, characterized in that the solvent constituting the oil phase is hydrogenated polyisobutene.

4. Composition as defined in claim 1, characterized in that the solvent constituting the oil phase is squalene.

5. Composition as defined in any one of claims 1 to 4, in which the emulsifying agent or agents of the water in oil type, are selected from sorbitan monooleate, sorbitan isostearate or sorbitan oleate ethoxylated with 5 moles of ethylene oxide.

6. Composition as defined in one of claims 1 to 5, in which the emulsifying agent or agents of the oil in water type are selected from sorbitan oleate ethoxylated with 20 moles of ethylene oxide, castor oil ethoxylated with 40 moles of ethylene oxide, sorbitan laurate ethoxylated wit 20 moles of ethylene oxide, lauric alcohol ethoxylated with 7 moles of ethylene oxide.

7. Composition as defined in any one of claims 1 to 6, in which the emulsifying agent or agents of the oil in water type are selected from compounds of the formula (I):
R₁—O—[CH(R₂)—CH₂—O]ₙ-(G)ₓ-H (I)
in which R₁ represents a linear or branched hydrocarbon radical, saturated or unsaturated, comprising 1 to 30 carbon atoms, R₂ represents a hydrogen atom or alkyl radical comprising 1 or 2 carbon atoms, G represents the rest of a saccharide, x represents a decimal number comprised between 1 and 5, and n is equal either to zero or to a whole number from 1 to 9.

8. Composition as defined in claim 7, for which, in the formula (I), x is comprised between 1 and 3, more particularly between 1.05 and 2.5, still more particularly between 1.1 and 2.0 and preferably less than or equal to 1.5.

9. Composition as defined in one of claims 7 or 8 for which, in formula (I), G represents the rest of the glucose or the rest of the xylose and n is equal to 0.

10. Composition as defined in any one of claims 7 to 9, for which, in the formula (I) R₁ represents a radical comprising 8 to 18 carbon atoms and more particularly an octyl, decyl, undecyl, dodecyl, tetradecyl or hexadecyl radical, said radicals being linear or branched.

11. Composition as defined in any one of claims 1 to 10 for which, the strongly acidic function of the monomer included in it is the sulfonic acid function or the phosphonic acid function partially or totally salified and preferably the monomer is 2-methyl 2-[(1-oxo 2-propenyl) amino] 1-propanesulfonic acid partially or totally salified in the form of an alkali metal salt such as for example the sodium or potassium salt, the ammonium salt, the salt of an amino alcohol such as for example the salt of monethanolamine or an amino acid salt such as for example lysine salt.

12. Composition as defined in any one of claims 1 to 11 for which, the weakly acidic function of the monomer included therein is the carboxylic acid function and preferably said monomer is selected from acrylic acid, methacrylic acid, itaconic acid or malic acid, partially or totally salified.

13. Composition as defined in any one of claims 1 to 12 for which the neutral monomer is selected from 2-hydroxy ethyl acrylate, 2,3-dihydroxy propyl acrylate, 2-hydroxy ethyl methacrylate, 2,3-dihydroxy propyl methacrylate, or an ethoxylated derivative of a molecular weight comprised between 400 and 1000, of each of these esters.

14. Composition as defined in claim 12, in which the polyelectrolyte is an acidic acid homopolymer partially or totally salified.

15. Composition as defined in claims 11 and 13, in which the polyelectrolyte is a copolymer comprising, in molar proportions, between 30% and 90% and more particularly between 50% and 90% of 2-methyl 2-[(1-oxo 2-propenyl)amino] 1-propane sulfonic acid partially or totally salified, and between 10 and 70% and more particularly between 10 and 50% of 2-hydroxy ethyl acrylate.

16. Composition as defined in claim 15, of 20 to 60% by weight and preferably 25 to 45% by weight of the copolymer.

17. Composition as defined in claims 15 or 16, in which the copolymer comprises, in molar proportions, from 60 to 90% of sodium salt or ammonium salt of 2-methyl 2-[(1-oxo 2-propenyl)amino] 1-propane sulfonic acid and from 10% to 40% of 2-hydroxy ethyl acrylate.

18. Composition as defined in claims 11 and 12, in which the polyelectrolyte is a copolymer comprising in molar proportions between 30 and 90% and more particularly between 30 and 45% of sodium salt, of ammonium salt, of monoethanolamine salt, or of lysine salt, of 2-methyl 2-[(1-oxo 2-propenyl)amino] 1-propane sulfonic acid and between 10 and 70% and more particularly between 55 and 70% of acrylic acid partially or totally salified, in the form of a sodium salt, the ammonium salt, the monoethanolamine salt or the lysine salt.

19. Composition as defined in any one of claims 1 to 18, characterized in that the polyelectrolyte is reticulated and/or branched with a diethylene or polyethylene compound, in the molar proportion expressed relative to the monomers used, of 0.005% to 1%, and preferably from 0.01% to 0.5% and more particularly from 0.1% to 0.25%.

20. Composition as defined in claim 19, characterized in that the reticulation and/or branching agent is selected from diallyoxyacetic acid or one of its salts such as sodium diallyloxyacetate, ethyleneglycol dimethacrylate, ethylene glycol diacrylate, diallyl urea, trimethylol propanetriacrylate, methylene bis(acrylamide), triallylamine or a mixture of these compounds.

21. Composition as defined in any one of claims 1 to 20, characterized in that it contains 4% to 10% by weight of emulsifying agents.

22. Composition as defined in claim 21, in which from 20% to 50% and more particularly from 25% to 40% of the total weight of the emulsifiers are of the water in oil type and from 80% to 50% and more particularly from 75 to 60% by weight are of the oil in water type.

23. Composition as defined in any one of claims 1 to 22, characterized in that the oil phase represents from 15% to 40%, and preferably from 20% to 25%, of its total weight.

24. Composition as defined in any one of claims 1 to 23, characterized in that it comprises moreover one or several additives selected from complexing agents, transfer agents or chain limiting agents.

25. Cosmetic, dermocosmetic, dermopharmaceutical or pharmaceutical composition, characterized in that it comprises from 0.1% to 10% by weight of the composition as defined in any one of claims 1 to 24.

26. Cosmetic, dermocosmetic, dermopharmaceutical or pharmaceutical composition as defined in claim 25, in the form of a milk, a lotion, a gel, a cream, a soap, a foaming bath, a balm, a shampoo or an after shampoo.

27. The use of a composition as defined in any one of claims 1 to 24, to prepare cosmetic, dermocosmetic, dermopharmaceutical or pharmaceutical compositions.
